(19) United States
(12) Patent Application Publication
(10) Pub. No.: US 2021/0290351 A1
(43) Pub. Date: Sep. 23, 2021

(54) METHOD FOR THE MANUFACTURE OF A BLANK AND BLANK

(71) Applicant: DENTSPLY SIRONA Inc., York, PA (US)

(72) Inventors: Lothar VOELKL, Goldbach (DE); Stefan FECHER, Johannesberg (DE); Martin KUTZNER, Neuberg (DE); Tanja OEFNER, Linsengericht (DE); Christian KOHLER, Hanau (DE); Tobias KRANZ, Reichenberg (DE); Andreas GEBHARDT, Langenselbold (DE); Martin HAIZMANN, Glauburg (DE); Heiner HOERHOLD, Büdingen (DE); Andrea BURNS, Hammersbach (DE)

(73) Assignee: DENTSPLY SIRONA Inc., York, PA (US)

(21) Appl. No.: 17/267,698

(22) PCT Filed: Aug. 1, 2019
(86) PCT No.: PCT/EP2019/070826

§ 371 Date: Feb. 10, 2021

(30) Foreign Application Priority Data
Aug. 17, 2018 (EP) 18189569.9

(51) Int. Cl. A61C 13/08; A61C 13/00; A61C 13/083; A61K 6/818; A61K 6/822; C04B 35/488; C04B 35/645

(52) U.S. Cl. CPC A61C 13/082; C04B 2235/765; A61C 13/083; A61K 6/818; A61K 6/822; C04B 35/4885; C04B 35/645; A61C 2201/002; C04B 2235/3225; C04B 2235/3272; C04B 2235/3277; C04B 2235/602; C04B 2235/75; C04B 2235/762; A61C 13/0022

(57) ABSTRACT

The present invention is related to a method for the manufacture of a multilayer blank, having a lowermost layer and a topmost layer, of a ceramic material, where layers of different compositions are introduced layer-by-layer into a mold, compressed and then sintered and where individual layers contain at least one coloring oxide, wherein as a first coloring oxide as the one coloring oxide at least one oxide from the group Co, Mn, Ni, Cr is used, the proportion of which in the lowermost layer containing the first coloring oxide is lower than in the topmost layer containing the first coloring oxide: and at least one oxide from the group Pr, Er, Fe, Ti, V, Bi, Cu, Tb is used as the second coloring oxide, the proportion of which in the lowermost layer containing the second coloring oxide is greater than that in the topmost layer.

### FIELD OF THE INVENTION

The present invention relates to a method for the manufacture of a multilayer blank, having a lowermost layer and a topmost layer, of a ceramic material as a base material or matrix material, in particular a blank to be used for the manufacture of a dental restoration, where layers of different compositions are introduced layer-by-layer into a mold, compressed and then sintered and where individual layers contain at least one coloring oxide and preferably at least two coloring oxides.

The invention also relates to a multi-layer blank for the manufacture of a dental restoration such as a crown, partial crown, bridge, veneer, abutment, in particular anatomical abutment, comprising at least one lowermost layer and one topmost layer of ceramic material of different compositions, where the ceramic material is the base material or matrix material of the respective layer.

The subject of the invention is also a dental restoration.

### BACKGROUND OF THE INVENTION

A corresponding method is disclosed in DE 10 2015 122 864 A1. To achieve a continuous transition between the individual layers, an intermediate layer is formed between the individual layers and contains a mixture of the materials of the adjacent layers. A dental restoration manufactured from a corresponding blank exhibits a continuously changing translucence commencing from the bottom layer.

EP 1 900 341 B1 discloses a multi-colored molded body which consists of a number of main layers of different colors between which are intermediate layers of different colors.

A blank according to WO2015/051095 A1 has at least two layers which differ both in color and translucence.

A block body of ceramic mass according to DE 10 2007 011 339 A1 has a number of layers, where in the transition region the gradient of change of the resulting optical properties is substantially constant.

Multilayer mold bodies are described in WO2010/010082 A1 and WO2015/052080 A1, where the layers have differing colors and translucencies.

A common feature of all of the multilayer blanks is that to attain different colorations/translucence properties, the coloring oxides used for this purpose decrease in quantity from the lowermost layer or bottom layer, i.e. root-side-extending layer towards the top, i.e. incisal-side-extending layer.

US 2017/0258563 A1 relates to a method for the production of a multilayer blank from ceramic material. As coloring oxide an element from the group comprising Co, Cr, Mn, Ni may be used.

US 201610000538 A1 refers to a method of manufacturing a zirconia block for the production of a dental prosthesis. The highest proportion of coloring oxide is in the lowermost layer.

A zirconia blank having several layers is disclosed in US 2016/0228223 A1. The layers made from zirconia include different mangane proportions with the layer provided for the incisal region having the largest mangane proportion and the subsequent layers having a smaller proportion.

DE 10 2015 102 864 A 1 relates to a method for the production of a blank having several ceramic layers with different proportions of coloring oxides.

### Objective of the Present Invention

The object of the present invention is to develop a method of the type mentioned at the outset such that a blank is made available from which a dental restoration can be made that can satisfy esthetic requirements and which corresponds or nearly corresponds to the natural appearance of teeth.

### SUMMARY OF THE INVENTION

To achieve this aim, the method described above is developed further such that as a first coloring oxide as the one coloring oxide at least one oxide from the group Co, Mn, Ni, Cr is used, the proportion of which in the lowermost layer containing the first coloring oxide being lower than that in the topmost layer containing the first coloring oxide: and at least one oxide from the group Pr, Er, Fe, Ti, V, Bi, Cu, Tb is used as the second coloring oxide, the proportion of which in the lowermost layer containing the second coloring oxide is greater than that in the topmost layer.

### BRIEF DESCRIPTION OF THE FIGURES

Further details, advantages and characteristics of the invention are provided not just by the claims, the characteristics to be drawn from them—alone and/or in combination—but also from the following description of the preferred embodiments shown in the drawing:

FIG. 1 shows—a schematic of a device for the manufacture of a green body,

FIG. 2 shows—a schematic of a blank, and

FIG. 3 shows—a schematic of a bridge to be manufactured from a blank

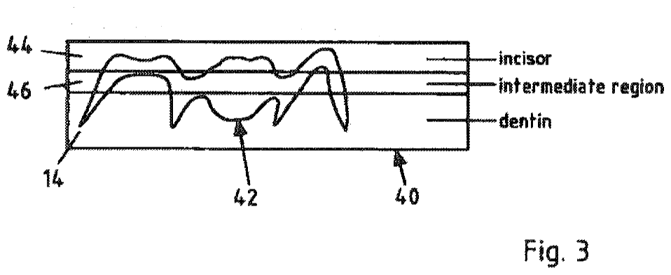

### DETAILED DESCRIPTION OF THE INVENTION

Layers preferably have at least two coloring oxides in the form of the first coloring oxide and a second coloring oxide, and as the second coloring oxide at least one oxide from the group Pr, Er, Fe, Ti, V, Bi, Cu, Tb is used, where its proportion in the lowermost layer is greater than in the topmost layer.

The topmost layer is that which contains the first coloring oxide. The content of the first coloring oxide in the topmost layer is higher than in the lowermost layer.

Usually the lowermost layer is the bottom layer and the topmost layer is the top layer.

In a departure from known methods, with which multi-layer blanks are manufactured, gray-coloring metallic oxides are used, the proportion of which is lowest in the lowermost layer and highest in the topmost layer, as a result of which in comparison to the prior art it surprisingly yields a more translucent and tooth-like appearance in the region of the cutting edge, which is the same or almost the same as that of natural teeth.

For the coloring itself, at least one second coloring oxide is used in the particular layer to attain a color that corresponds to that of one tooth or a group of teeth, i.e. a bridge. The proportion of the second coloring oxide here decreases from layer to layer, commencing from the lowermost layer, i.e. the layer extending on the root side.

In particular, at least one of the metal oxide powders from the group Al₂O₃, TiO₂, zirconium oxide mixed crystal Zr₁₋ₓMeₓO₂-(4n/2)ₓ is used for the ceramic material, or as the ceramic material, which is the base material or matrix material of the corresponding layer, where Me is a metal that in oxide form is present as a bivalent, trivalent or tetravalent cation (n=2, 3, 4 and 0≤x≤1) and the tetragonal and/or the cubic phase of the zirconium oxide, where in particular yttrium oxide-stabilized zirconium dioxide is used.

Al₂O₃ is also emphasized as a base or matrix material of the ceramic material.

Yttrium oxide-stabilized zirconium dioxide is in particular used as ceramic material, which with respect teethe individual layers can differ in its composition regardless of the added coloring oxides, for example to achieve desired strength values.

To stabilize the crystal phase in zirconium oxide at room temperature in addition to or as an alternative to Y₂O₃, at least one oxide from the group MgO, CaO, CeO₂, ScO₃ and YbO₃ can be used.

If Y₂O₃ is used, then it should be present in a percentage by weight of 4.5 to 13.0.

A color-intensive and naturally-appearing dental prosthesis can be made from a corresponding blank, in particular in the tooth neck region and in the dentine body. At the same time, a natural incisal region is created through the higher proportion, in comparison to the lowermost layer, of the first color oxide in the topmost layer, i.e. in the incisal region.

It was surprisingly found that, irrespective of the gray coloration of the first coloring oxide, an incisal region appears brighter and more translucent compared to a dental prosthesis in which the first coloring oxide according to the prior art decreases in proportion from the bottom or lowermost layer to the top or topmost layer.

It is in particular provided that after introduction of a layer into the mold, the surface of the layer is smoothed and a further layer is then introduced and the procedure is continued according to the number of layers.

Following introduction of the individual layers, these are then compressed as a unit.

To obtain a continuous transition between the individual layers, it is in particular provided that the smoothed surface of the last layer is structured to form elevations and depressions before introduction of a further layer.

The first coloring oxide is preferably an oxide from the group Co, Klin or mixtures thereof.

The subject of the invention is also a multi-layer blank for the manufacture of a dental restoration such as a crown, partial crown, bridge, veneer, abutment, in particular anatomical abutment, as an abutment having a crown, comprising at least one lowermost layer and one topmost layer of one or more ceramic materials of different compositions, where the layers contain at least one first coloring oxide, the proportion of which in the lowermost layer containing the second coloring oxide is greater than that in the topmost layer.

Furthermore, at least some of the layers should have a second coloring oxide which is preferably selected from the group Pr, Er, Fe, Ti, V, Bi, Cu, Tb. The proportion of the second coloring oxide in the lowermost layer is here greater than that in the topmost layer according to the prior art.

In particular, the proportion of the first coloring oxide increases from layer to layer commencing from the lowermost layer, and the proportion of the second coloring oxide decreases from layer to layer commencing from the lowermost layer or from the layer that contains the second coloring oxide.

There is naturally no departure from the invention if, for example, the topmost layer does not contain a second coloring oxide, or if sequential layers of a multilayer blank all have the same proportions of the coloring oxides. Irrespective thereof, the proportion of the first coloring oxide in the lowermost layer containing a first coloring oxide is lower than that in the topmost layer containing a first coloring oxide.

The invention is in particular characterized in that the proportion A1 of the first s coloring oxide in the lowermost layer is 0 ppm<A1≤100 ppm and/or the proportion A2 of the first coloring oxide in the topmost layer is 10 ppm≤A2≤200 ppm, where A2>A1.

In particular the invention provides for the proportion of the first coloring oxide to continuously change from layer to layer.

The number of layers can be selected according to the prior art. In particular, two to seven layers are provided, preferably three or four layers.

The invention is characterized by a dental restoration such as a crown, partial crown, bridge, veneer, abutment, in particular an anatomical abutment, in that the restoration has at least one root-side-extending lowermost layer and a incisal edge-side-extending topmost layer and preferably at least one intermediate layer extending between these, where the layers contain at least one first coloring oxide, contained in the lowermost layer in a lower proportion than in the topmost, layer. The first coloring oxide is here in particular at least one oxide from the group Co, Mn, Mi, Cr, where Co₃O₄ or MnO₂ or mixtures thereof are preferred.

The dental restoration should further contain a second coloring oxide from the group Pr, Er, Fe, Ti, V, Bi, Cu, Tb, where the proportion in the lowermost layer is greater than in the topmost layer.

The dental restoration should in particular contain as ceramic material yttrium oxide (Y₂O₃), calcium oxide (CaO), magnesium oxide (MgO) and/or cerium oxide (CeO₂) and/or scandium oxide (ScO₃) and/or ytterbium oxide (YbO₃), in particular zirconium oxide to which yttrium oxide is added, where the material of the lowermost layer differs from the material of the topmost layer in color and/or proportions of crystal forms stabilized at room temperature.

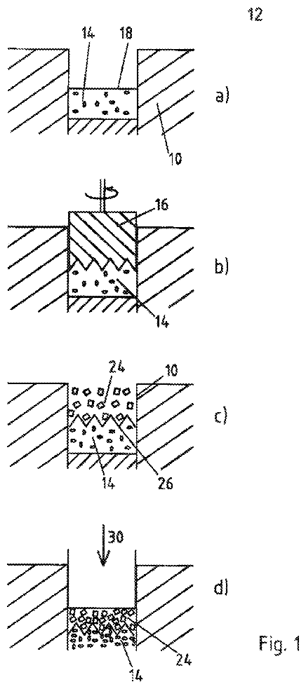

Turning now to the Figures, by reference to FIG. 1, it will be made clear purely in principle how a blank for a dental restoration is to be produced. Thus, in accordance with FIG. 1a a first ceramic material is filled into a mold 10 of a press, in the embodiment an yttrium oxide-stabilized zirconium dioxide as the base material or matrix material, which can have the following composition in percentage by weight:

HfO₂<3.0,

Al₂O₃<0.3.

Technically unavoidable impurities <0.2 (such as SiO₂, Na₂O)
Y₂O₃4.5 to 13.0
Co₃O₄ as the first coloring oxide >0 ppm to 200 ppm
Fe₂O₃ and Er₂O₃ as the second coloring oxide 2,000 ppm to 8,000 ppm

ZrO₂=100%−(Y₂O₃+Al₂O₃+HfO₂+unavoidable impurities+

coloring first oxide+coloring second oxides)

After introduction of the first layer 14, its surface is smoothed (FIG. 1a) and then structured (FIG. 1b). For this purpose, in accordance with the drawing a flat element 16 having an edge with a zig-zag geometry can be used, which penetrates the surface 18 while rotating to form a structure 26 which due to elevations and depressions, i.e. due to the element 16, consists of peaks and valleys extending is in concentric circles.

The proportion of the first coloring oxide can, according to an embodiment, be in the range 3-8 ppm, and that of the second coloring oxide or the second coloring oxides can be between 6,500 ppm and 7,000 ppm to produce, for example, a tooth of VITA color A3.

A second ceramic material 24 is then introduced into the mold 10 (FIG. 1c), which with the exception of the first and second coloring oxides can correspond to or differ from that of the first layer 14. In particular, it is provided that the material of the second layer differs from the ceramic material of the first layer 14 in terms of yttrium oxide proportion. There is also a difference in the first coloring oxide and the second coloring oxide. The proportion of the first coloring oxide in the form of Co₃O₄ is greater than in the first layer 14. In particular, the proportion of CO₃O₄is in the range 15-20 ppm. The proportion of the second coloring oxide decreases in comparison to the first layer 14 and can be in the range 5,500 to 6,000 ppm.

In addition, the proportion of yttrium oxide increases from the lowermost or bottom layer to the topmost or top layer.

With regard to the ranges of values, it is to be noted that according to the teaching of the invention, the first coloring oxide in the second layer is proportionately greater than in the first layer 14, and conversely, the second coloring oxides are proportionately lower in the second layer 24 than in the first layer 14.

The surface of the second layer 24 is then smoothed and structured so that corresponding further layers can be applied, the proportions of oxides being selected in accordance with the teaching of the invention such that the first coloring oxide increases and the proportion of the second coloring oxides decreases.

In the topmost or top layer, the proportion of the first coloring oxide, for example, which in the embodiment is Co₃O₄, can be between 35 ppm and 45 ppm, and the proportion of the second coloring oxides can be between 4,000 ppm and 5,000 ppm.

Once all the layers have been introduced, they are compressed in the matrix as a unit (FIG. 1d). The compressing (arrow 30) can be carried out at a pressure between, for example, 1,000 bar and 2,000 bar. Pre-sintering is then carried out at a temperature between, for example, 800° C. and 1,000° C. for a time period between, for example, 100 minutes and 150 minutes.

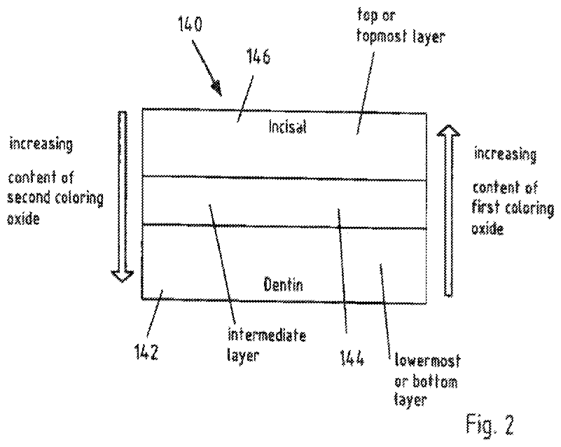

A corresponding blank 140 exhibits, as a result of the coloring oxides added to the ceramic material, a gradation in color intensity and translucence, as shown in FIG. 2. In the embodiment, the blank 140 has a bottom or lowermost layer 142, an intermediate layer 144 and a top or topmost layer 146. The proportion of the first coloring oxide, such as Co₃O₄, increases from the bottom layer 142 in the direction of the top layer 146. By contrast, the proportion of the second coloring oxide decreases from the bottom layer (lowermost layer 142) in the direction of the top layer (topmost layer 146).

Whilst the proportion of gray cobalt oxide increases from the bottom layer in the direction of the top layer, i.e. from the dentine region to the incisal region, it was surprisingly found that a translucence can be achieved which corresponds in the cutting region of the dental prosthesis to be manufactured from the blank to that of a natural tooth, and surprisingly the gray cobalt oxide makes the incisal region brighter and more translucent.

A dental prosthesis, such as a bridge 42, can now be produced from a corresponding blank 40 in a CAD/CAM process. For this purpose, a milling program is designed such that the lower region of the bridge 42 extends in the region of the first or bottom layer 14 and the incisal region 44 extends in the top layer. The dentine region extends in the bottom region 14 and the incisal region extends in the top region with a translucence that is very close to that of a natural tooth.

If layers of zirconium oxide that differ in terms of yttrium oxide content are used, then the strength can additionally be influenced. Thus, the yttrium oxide proportion in the top layer can be chosen such that the proportion of the cubic crystal phase after pre-sintering is considerably greater than that in the bottom layer 14, in which the tetragonal crystal phase should be more than 90%. In the top layer, the cubic crystal phase proportion should be between 30% and 49%. The remainder is substantially the tetragonal crystal phase.

These different crystal phases are achieved in that in the bottom layer the yttrium oxide proportion is in the range 4.5 to 7 percent by weight, and in the top s layer is in the range 7 to 13.0 percent by weight, where the proportion in the first layer 14 is lower than in the top layer.

1. A method for the manufacture of a multilayer blank comprising the steps of:
introducing lavers of different ceramic compositions layer-by-layer into a mold,
compressing the layers of different ceramic compositions; and
sintering the compressed layers of different ceramic compositions; and
wherein the layers include a topmost layer and a lowermost layer, each having at least one coloring oxide,
wherein the at least one coloring oxide includes a first coloring oxide being selected from the group consisting of Co, Mn, Ni, and Cr;
wherein the lowermost layer includes the first coloring oxide in a lower proportion than that of the topmost layer having the first coloring oxide;
wherein the at least one coloring oxide includes a second coloring oxide being selected from the group consisting of Pr, Er, Fe, Ti, V, Bi, Cu, and Tb; and
wherein the lowermost layer includes the second coloring oxide in a greater proportion than that of the topmost layer having the second coloring oxide.

2. The method according to claim 1, wherein after introduction of a layer into the mold a surface of the layer is smoothed and a further layer is introduced, and the procedure is continued according to the number of layer.

3. The method according to claim 1, wherein an oxide being selected from the group of the first coloring oxide consisting of Co, Mn and mixtures thereof is used as the first coloring oxide.

4. The method according to claim 1, wherein at least one of the different ceramic compositions includes at least one metal oxide powder being selected from the group consisting of Al₂O₃, TiO₂, and zirconium oxide mixed crystal Zr₁₋ₓMeₓO₂-(4n/2)ₓ, where Me is a metal that in oxide form is present as a bivalent, trivalent or tetravalent cation (n=2, 3, 4 and 0≤x≤1) and the tetragonal and/or cubic phase of the zirconium oxide.

5. The method according to claim 1, wherein the lowermost layer is used for a restoration to be manufactured from the blank for the dentine region, and the topmost layer for the incisal region.

6. A multi-layer blank for the manufacture of a dental restoration comprising a plurality of layers of different ceramic compositions, the plurality of layers including a lowermost layer and a topmost layer;
wherein the lowermost layer and the topmost layer includes at least one first coloring oxide, such that the lowermost layer includes the at least one first coloring oxide in as lower proportion than the first coloring oxide in the topmost layer,
wherein the at least one first coloring oxide being selected from the group consisting of Co, Mn, Ni, Cr, or and a mixture thereof.

7. The blank according to claim 6, wherein the proportion of the at least one first coloring oxide increases from layer to layer, commencing from the lowermost layer.

8. The blank according to claim 6, wherein a proportion A1 of the at least one first coloring oxide in the lowermost layer is 0 ppm<A1≤100 ppm.

9. The blank according to claim 6, wherein the number of layers is between 2 and 7.

10. The blank according to claim 6, wherein the lowermost layer and/or the topmost layer include a base material having aluminum oxide or yttrium oxide-stabilized zirconium oxide.

11. Dental restoration, manufactured from a blank according to claim 6, wherein the restoration viewed in a tooth axis direction comprises a lowermost layer extending on the root side, a top layer extending on the incisal side, and at least one intermediate layer extending therebetween, wherein the at least one first coloring oxide in the lowermost layer is in a lower proportion than the at least one first coloring oxide in the topmost layer.

12. The dental restoration according to claim 11, wherein the at least one first coloring oxide being selected from the group consisting of Co, Mn, Ni, Cr, and a mixture thereof.

13. The dental restoration according to claim 11, wherein the base material of the restoration is aluminum oxide or yttrium oxide-stabilized zirconium dioxide.

14. The method of claim 2, wherein before introduction of a further layer, the smoothed surface of the previous layer is structured to form elevations and depressions such that after the introduction of all layers into the mold, the layers are then compressed as a unit.

15. The blank according to claim 6, further comprising at least one second coloring oxide being selected from the group consisting of Pr, Bi, Er, Fe, Ti, V, Cu, and Tb, wherein a proportion of the at least one second coloring oxide in the lowermost layer is greater than the at least one second coloring oxide in the topmost layer.

16. The black according to claim 7, wherein a proportion of the at least one second coloring oxide decreases from layer to layer; commencing from the lowermost layer.

17. The black according to claim 8, wherein a proportion A2 of the first coloring oxide in the topmost layer is 10 ppm≤A2≤200 ppm, and where A2>A1.

18. The black according to claim 10, wherein the base material includes yttrium oxidestabilized zirconium oxide in an amount in the range of 4.5 to 13.0 percent by weight and wherein a proportion of yttrium oxide-stabilized zirconium oxide increases from the lowermost layer to the topmost layer.

19. The dental restoration according to claim 12, wherein the lowermost layer and the top layer include at least one second coloring oxide being selected from the group consisting of Pr, Er, Fe, Ti, V, Bi, Cu, and Tb, such that a proportion of the at least one second coloring oxide is higher in the lowermost layer than in the topmost layer.

20. The dental restoration according to claim 13, wherein the base material includes yttrium oxide-stabilized zirconium oxide in an amount in the range of 4.5 to 13.0 percent by weight and wherein a proportion of yttrium oxide-stabilized zirconium oxide increases from the lowermost layer to the topmost layer.
